(19) United States
(12) Patent Application Publication
(10) Pub. No.: US 2026/0184694 A1
(43) Pub. Date: Jul. 2, 2026

(54) COMPOSITION COMPRISING HIGH PURITY TRIAMINOPYRIMIDINE COMPOUND AND METHOD FOR PREPARATION THEREOF

(71) Applicant: ZYDUS LIFESCIENCES LIMITED, Ahmedabad (IN)

(72) Inventors: Kumar Kamlesh SINGH, Ahmedabad (IN); Nikhil Amar SINGH, Ahmedabad (IN); Hardik Bhikhubhai GHODASARA, Ahmedabad (IN); Samir Rameshbhai GAJJAR, Ahmedabad (IN); Jaydeep Nanjibhai LALPARA, Ahmedabad (IN); Sandip Kiritbhai UPADHYAY, Ahmedabad (IN); Neetandra Shyam Bahadur SINGH, Ahmedabad (IN)

(21) Appl. No.: 19/436,269

(22) Filed: Dec. 30, 2025

(30) Foreign Application Priority Data
Dec. 30, 2024 (IN) 202421104622

(51) Int. Cl. C07D 401/14

(52) U.S. Cl. CPC C07D 401/14

(57) ABSTRACT

The present invention relates to a composition comprising high purity triaminopyrimidine compound of Formula (I) and method for preparation thereof. The present invention particularly relates to compositions comprising triaminopyrimidine compound having purity of about 99.0% or more when measured by area percentage of high-performance liquid chromatography (HPLC), and one or more of compounds of Formula (II), Formula (III), and Formula (IV) as described herein, wherein each of the compounds of Formulae (II), (III) or (IV), is present in an amount of about 0.15% or less by weight, relative to compound of Formula (I), when measured by HPLC. The present invention also provides the triaminopyrimidine compound of Formula (I) substantially free of compounds of Formula (V) or Formula (VI) as described herein.

### FIELD OF THE INVENTION

The present invention relates to a composition comprising a high purity triaminopyrimidine compound of Formula (I) and method for preparation thereof. The present invention particularly relates to compositions comprising a triaminopyrimidine compound having a purity of about 99.0% or more, when measured by area percentage of high performance liquid chromatography (HPLC), and one or more of compounds of Formula (II), Formula (III), and

Formula (IV), wherein each of the compounds of Formula (II), Formula (III), or Formula (IV) is present in an amount of about 0.15% or less by weight, relative to the compound of Formula (I), when measured by HPLC. The present invention also relates to the triaminopyrimidine compound of Formula (I) substantially free of compounds of Formula (V) or Formula (VI).

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

International (PCT) Publication No. WO 2015/165660 A1 (the WO '660) discloses triaminopyrimidine compounds, intermediates, pharmaceutical compositions and methods for use for preventing or treating malaria, wherein one of the triaminopyrimidine compound disclosed is a compound of Formula (I) as represented below:

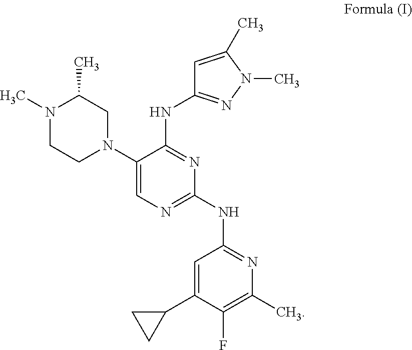 [SMILES: Cc1nc(Nc2ncc(N3CCN(C)[C@H](C)C3)c(Nc3cc(C)n(C)n3)n2)cc(C2CC2)c1F]

The WO '660 discloses a process for the preparation of compound of Formula (I) and intermediates thereof.

Nature Communications, 6 (1) (2015): 6715, also discloses a process for the preparation of a triaminopyrimidine compound of Formula (I), and its use for treating malaria.

International (PCT) Publication No. WO 2019/049021 A1 (WO '021) discloses a process for the preparation of compound of Formula (I) and crystalline Form I and Form II of the compound of Formula (I).

In a pharmaceutical product, an impurity is first and foremost, a quality issue, since it could potentially compromise the efficacy of the drug product. Secondly, impurities also cause safety concerns. Therefore, the objective is to know the plausible impurities for allowing assessment of their toxicological implications and for understanding their formation mechanisms, which is an important knowledge for improving the synthetic chemical pathway and optimizing the formulation.

In view of the above, it is therefore desirable to provide a composition comprising triaminopyrimidine compound of Formula (I) having higher purity and substantially free of impurities.

### SUMMARY OF THE INVENTION

In one general aspect, the present invention provides a compound selected from a compound of Formula (II), a compound of Formula (III), or a compound of Formula (IV),

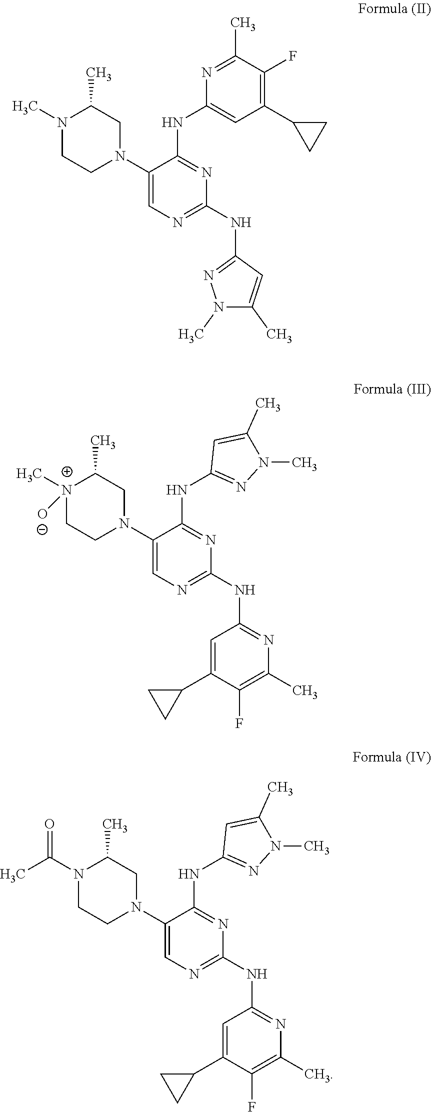

In another general aspect, the present invention provides a composition comprising a triaminopyrimidine compound of Formula (I)

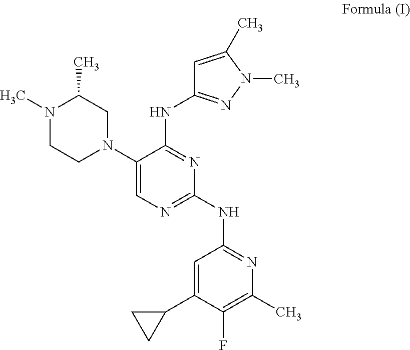

having a purity of about 99.0% or more, when measured by area percentage of high performance liquid chromatography (HPLC), and one or more of compounds of Formula (II), Formula (III), and Formula (IV), wherein each of the compound of Formula (II), Formula (III), or Formula (IV) is present, in an amount of about 0.15% or less by weight, relative to the compound of Formula (I), when measured by HPLC,

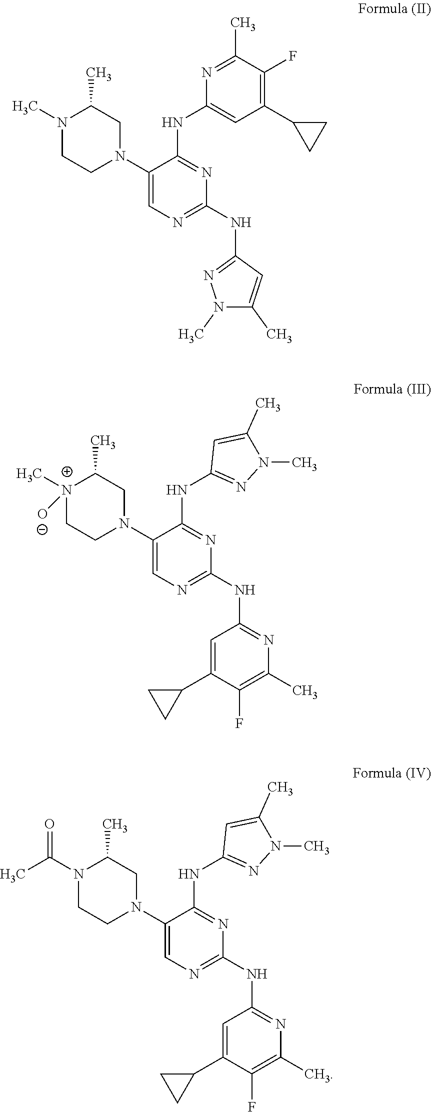

In another general aspect, the present invention provides a composition comprising a triaminopyrimidine compound of Formula (I) having a purity of 99.0% or more, when measured by area percentage of HPLC, and one or more compounds of Formula (V), or Formula (VI), each present, in an amount of about 500 ppm or less, when measured by HPLC,

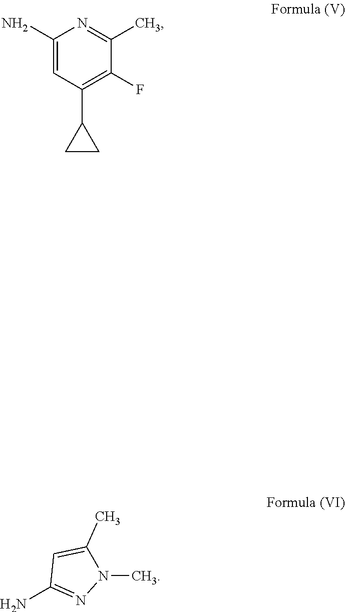

In another general aspect, the present invention provides a stable composition comprising a triaminopyrimidine compound of Formula (I), having a purity of about 99.0% or more, when measured by area percentage of HPLC, and one or more of compounds of Formula (II), Formula (III), Formula (IV), Formula (V), and Formula (VI), wherein each of the compounds of Formula (II), Formula (III), or Formula (IV), is present, in an amount of about 0.15% or less, by weight, relative to the triaminopyrimidine compound of Formula (I), when measured by HPLC, and each of the compounds of Formula (V), or Formula (VI) is present in an amount of about 134 ppm or less, by weight, relative to the compound of Formula (I), when measured by HPLC, when the composition is stored for at least 6 months at 40° C.±2° C. temperature and 75%+5% relative humidity, or at 25° C.±2° C. temperature and 60±5% relative humidity.

In another general aspect, the present invention provides a triaminopyrimidine compound of Formula (I) having a purity of about 99.0% or more, when measured by area percentage of HPLC and is substantially free of one or more of compounds of Formula (II), Formula (III), Formula (IV), Formula (V) or Formula (VI).

In another general aspect, the present invention provides a triaminopyrimidine compound of Formula (I), having a purity of about 99% or more as measured by area percentage of HPLC, and containing:
(i) a compound of Formula (II), in an amount of about 0.15% or less,
(ii) a compound of Formula (III), in an amount of about 0.15% or less,
(iii) a compound of Formula (IV), in an amount of about 0.15% or less, by weight relative to the compound of Formula (I), when measured by HPLC; or
(iv) a compound of Formula (V), in an amount of about 500 ppm or less, or
(v) a compound of Formula (VI), in an amount of about 500 ppm or less, when measured by HPLC.

In another general aspect, the present invention provides a process for the preparation of a triaminopyrimidine compound of Formula (I),

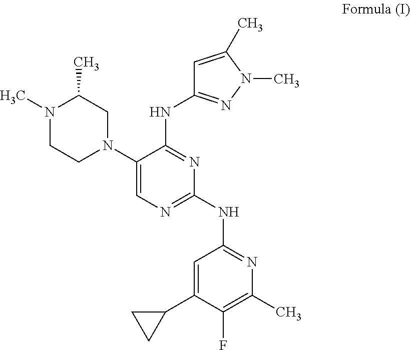

having a purity of about 99.0% or more, when measured by area percentage of HPLC, and substantially free of one or more compounds of Formula (II), Formula (III), Formula (IV), Formula (V), or Formula (VI), the process comprising:
(a) reacting a compound of Formula (X),

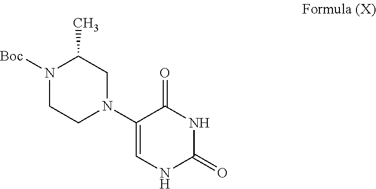 [SMILES: C[C@@H]1CN(c2c[nH]c(=O)[nH]c2=O)CCN1C(=O)OC(C)(C)C]

with phosphorus oxychloride at a temperature of about 100° C. to about 115° C., to obtain a compound of Formula (IX), or a salt thereof,

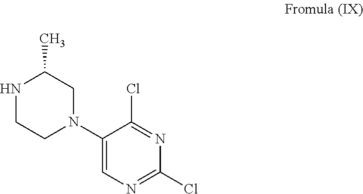

(b) reacting the compound of Formula (IX), or a salt thereof, with a methylating agent, in the presence of a reducing agent, in one or more solvents at a temperature of about 10° C. to about 30° C., to obtain a compound of Formula (VII), or a salt thereof,

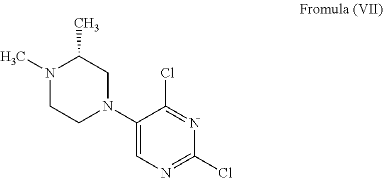 [SMILES: C[C@@H]1CN(c2cnc(Cl)nc2Cl)CCN1C]

(c) reacting the compound of Formula (VII), or a salt thereof, with a compound of Formula (VI), or a salt thereof, in the presence of one or more bases in one or more solvents, at a temperature of about 90° C. to about 120° C.,

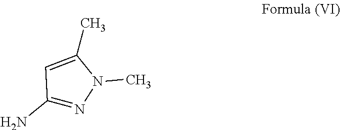

to obtain a compound of Formula (VIII), or a salt thereof,

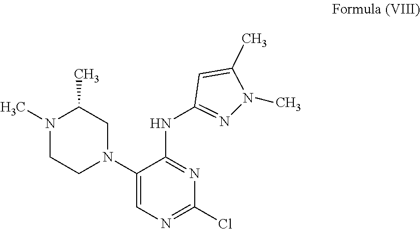

(d) reacting the compound of Formula (VIII), or a salt thereof, with a compound of Formula (V), or a salt, or a hydrate thereof, in the presence of a Grignard reagent used in an amount of about 3 to about 6 mole equivalents relative to the compound of Formula (VIII), in one or more solvents,

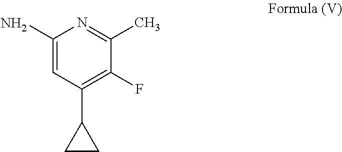

to obtain the compound of Formula I, and
(e) crystallizing the compound of Formula (I) in one or more solvents to obtain the compound of Formula (I) having a purity of about 99.0% or more, when measured by area percentage of HPLC and substantially free of one or more compounds of Formula (II), Formula (III), Formula (IV), Formula (V), or Formula (VI).

In another general aspect, the present invention provides a pharmaceutical composition comprising a triaminopyrimidine compound of Formula (I) having a purity of about 99.0% or more, when measured by area percentage of HPLC and substantially free of one or more compounds of Formula (II), Formula (III), Formula (IV), Formula (V), and Formula (VI), together with one or more pharmaceutically acceptable excipients.

The details of one or more embodiments of the invention are set forth in the accompanying description.

### DETAILED DESCRIPTION OF THE INVENTION

The aforementioned general and further specific aspects of the invention are fulfilled by the description of the invention provided herein after.

The invention can further be understood in light of the description of the embodiments provided herein after. It is to be understood that the description, in no way, is intended to limit the scope of the invention to the expressly specified embodiments only. The equivalents and variants thereof or trivial modifications thereof which are apparently obvious to those skilled in the art, are also intended to be included within the scope of the present invention.

Detailed description of routine and conventional unit operations, which are easily understood by the skilled artisan, are not included herein. Such routine unit operations are to be construed as ordinarily understood and as routinely practiced by the person skilled in the field of the invention, unless otherwise specifically described.

The following definitions are used in connection with present application, unless it is indicated otherwise.

The term “composition” used herein means a physical mixture of two or more components.

The term “in a detectable amount”, refers to the level of impurity in the product, which is in the level of detection limit, when analyzed using the HPLC method.

The term “non-detectable amount” or “not in a detectable amount”, refers to the level of impurity in the product, which is below the level of detection limit, when analyzed using the HPLC method.

All ranges recited herein include the endpoints, including those that recite the range “between” two values.

Terms such as “about” and the like are to be construed as modifying a term or value such that it is not an absolute. This includes, at very least, the degree of expected experimental error, technique error and instrument error for a given technique used to measure a value.

The phrase “substantially free of one or more compounds of Formula (II), Formula (III), Formula (IV), Formula (V), or Formula (VI)”, as used herein refers to the compound of Formula (I) containing one or more impurities of compounds of Formula (II), Formula (III), or Formula (IV) in an amount within the permissible ICH limits suitable for pharmaceutical preparations, for example, but not limited to about 0.15% or less, or to about 0.11% or less, or to about 0.1% or less, or more particularly to about 0.05% or less, or most particularly not in a detectable amount, by weight, relative to the compound of Formula (I), when measured by HPLC, or absent, and the compound of Formula (V) or Formula (VI) in an amount of about 500 ppm or less, or about 400 ppm or less, or about 300 ppm or less, about 200 ppm or less, or about 134 ppm or less, or about 100 ppm or less, or about 50 ppm or less, or not in a detectable amount, when measured by HPLC, or absent.

The phrase “wherein each of the compound of Formula (II), Formula (III), or Formula (IV) is present, in an amount of about 0.15% or less” means each of the compound of Formula (II), Formula (III), or Formula (IV) is present in the composition in an amount of about 0.15% or less, or about 0.11% or less, about 0.1% or less, or about 0.05% or less, or about 0.05% or less, or not in a detectable amount, by weight relative to compound of Formula (I), when measured by area percentage of HPLC, or absent.

Alternatively, the phrase “wherein each of the compound of Formula (II), Formula (III), or Formula (IV) is present, in an amount of about 0.15% or less” refers to each of the compound of Formula (II), Formula (III), or Formula (IV), when present in the composition, is present in a detectable amount of about 0.15% or less, by weight relative to the compound of Formula (I) by weight, when measured by HPLC.

The phrase “wherein each of the compound of Formula (V), or Formula (VI) is present, in an amount of about 500 ppm or less” means each of the compound of Formula (V), or Formula (VI) is present in the composition in an amount of about 500 ppm or less, or about 400 ppm or less, or about 300 ppm or less, or about 200 ppm or less, or about 150 ppm or less, or about 134 ppm or less, or about 100 ppm or less, or about 50 ppm or less, or not in a detectable amount, when measured by HPLC, or absent.

Alternatively, the phrase “wherein each of the compound of Formula (V), or Formula (VI) is present, in an amount of about 500 ppm or less” refers to each of the compound of Formula (V) or Formula (VI), when present in the composition, is present in a detectable amount of about 500 ppm or less, when measured by HPLC.

The term “impurity” includes one or more degradants which arise during storage of the compound of Formula (I) and/or one or more by-products formed in a chemical reaction used in manufacturing of the compound of Formula (I). In some embodiments, an impurity results from oxidation, light-initiated decomposition, reaction with a residual solvent, a side reaction that takes place during the process used to prepare the compound of Formula (I), or a reaction of compound of Formula (I).

The term “storage” refers to storage for up to 6 months and different stability conditions refers to storage at 25° C.±2° C. temperature and 60±5% relative humidity; or at 40° C.±2° C. temperature and 75±5% relative humidity.

The term “excipient(s)” or “pharmaceutically acceptable excipient(s)” refers to pharmacologically inactive substances that are added to a pharmaceutical preparation in addition to the active pharmaceutical ingredient. The pharmaceutically acceptable excipients may include one or more diluents, binders, disintegrants, lubricants, glidants, sweeteners/taste masking agents, colorants, flavours, film-forming agents, plasticizers and the like.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and one or more pharmaceutically acceptable excipients.

The control of genotoxic impurities (GTIs) is a crucial activity that is performed for any new chemical entity intended for clinical use. The compound of Formula (V) and compound of Formula (VI) are known intermediates used for the synthesis of the compound of Formula (I). The present inventors surprisingly found that the compound of Formula (V) and Formula (VI) are potential genotoxic impurities present in the compound of Formula (I) as predicted by Derek. The present inventors were able to obtain the compound of Formula (I) substantially free of compound of Formula (V) and Formula (VI) as per the ICH guidelines.

Thus, in one general aspect, the present invention provides a compound selected from a compound of Formula (II), a compound of Formula (III), or a compound of Formula (IV).

In one embodiment, the present invention provides the impurities selected from compound of Formula (II), Formula (III), or Formula (IV) as a “reference marker” and/or “reference standard” in methods for determining the identity and/or the amount of said impurities in a sample of the compound of Formula (I).

In another general aspect, the present invention provides a composition comprising a triaminopyrimidine compound of Formula (I) having a purity of about 99.0% or more, when measured by area percentage of HPLC, and one or more of compounds of Formula (II), Formula (III), and Formula (IV), wherein each of the compounds of Formula (II), Formula (III), or Formula (IV) is present, in an amount of 0.15% or less by weight, relative to the compound of Formula (I), when measured by HPLC.

In another embodiment, the present invention provides a composition comprising a triaminopyrimidine compound of Formula (I) having a purity of about 99.0% or more, when measured by area percentage of HPLC, and one or more of compounds of Formula (II), Formula (III), and Formula (IV), wherein each of the compounds of Formula (II), Formula (III), or Formula (IV), is present, in an amount of 0.11% or less by weight, relative to the compound of Formula (I), when measured by HPLC.

In another embodiment, each of the compounds of Formula (II), Formula (III), or Formula (IV) is present in the composition in a detectable amount by weight, when measured by HPLC.

In another embodiment, the present invention provides a composition comprising a triaminopyrimidine compound of Formula (I) having a purity of about 99.0% or more, when measured by area percentage of HPLC, for example about 99.5% or more, or about 99.8% or more, when measured by area percentage of HPLC.

In another embodiment, the present invention provides a composition comprising a triaminopyrimidine compound of Formula (I) having a purity of about 99.0% or more, when measured by area percentage of HPLC, and one or more of compounds of Formula (II), Formula (III), and Formula (IV), wherein each of the compounds of Formula (II), Formula (III), or Formula (IV), is present in an amount in the range of about 0.001% to about 0.15% by weight, relative to the compound of Formula (I), when measured by HPLC.

In another embodiment, each of the compounds of Formula (II), Formula (III), or Formula (IV), is present in the composition in an amount in the range of about 0.005% to about 0.15% by weight, relative to the compound of Formula (I), when measured by HPLC.

In another embodiment, each of the compounds of Formula (II), Formula (III), or Formula (IV), is present in the composition in an amount in the range of about 0.01% to about 0.15% by weight, relative to the compound of Formula (I), when measured by HPLC.

In another embodiment, each of the compounds of Formula (II), Formula (III), or Formula (IV), is present in the composition in an amount in the range of about 0.001% to about 0.11% by weight, relative to the compound of Formula (I), when measured by HPLC.

In another embodiment, each of the compounds of Formula (II), Formula (III), or Formula (IV), is present in the composition in an amount in the range of about 0.005% to about 0.11% by weight, relative to the compound of Formula (I), when measured by HPLC.

In another embodiment, the present invention provides a composition comprising a triaminopyrimidine compound of Formula (I) having a purity of about 99.0% or more, when measured by area percentage of HPLC, and one or more of compounds of Formula (II), Formula (III), and Formula (IV), wherein each of the compounds of Formula (II), Formula (III), or Formula (IV) is present, in an amount of upto 0.15% by weight, relative to the compound of Formula (I), when measured by HPLC.

In another embodiment, the present invention provides a composition comprising a triaminopyrimidine compound of Formula (I) having a purity of about 99.0% or more, when measured by area percentage of HPLC, and one or more of compounds of Formula (II), Formula (III), and Formula (IV), wherein each of the compounds of Formula (II), Formula (III), or Formula (IV) is present, in an amount of upto 0.11% by weight, relative to the compound of Formula (I), when measured by HPLC.

In another embodiment, the present invention provides a composition comprising a triaminopyrimidine compound of Formula (I) having a purity of about 99.0% or more, when measured by area percentage of HPLC, and a compound Formula (II), Formula (III), and Formula (IV), wherein each of the compounds of Formula (II), Formula (III), or Formula (IV) is present, in an amount of 0% to about 0.15% by weight, relative to the compound of Formula (I), when measured by HPLC.

In another embodiment, the present invention provides a composition comprising a triaminopyrimidine compound of Formula (I) having a purity of about 99.0% or more, when measured by area percentage of HPLC, and a compound Formula (II), Formula (III), and Formula (IV), wherein each of the compounds of Formula (II), Formula (III), or Formula (IV) is present, in an amount of 0% to about 0.11% by weight, relative to the compound of Formula (I), when measured by HPLC.

In another embodiment, each of the compounds of Formula (II), Formula (III), and Formula (IV) is present in the composition in an amount in the range of about 0.001% to about 0.11% by weight, relative to the compound of Formula (I), when measured by HPLC.

In yet another embodiment, each of the compounds of Formula (II), Formula (III), and Formula (IV) is present in the composition in an amount in the range of about 0.01% to about 0.11% by weight, relative to the compound of Formula (I), when measured by HPLC.

In yet another embodiment, each of the compounds of Formula (II), Formula (III), and Formula (IV) is present in the composition in an amount in the range of about 0.001% to about 0.08% by weight, relative to the compound of Formula (I), when measured by HPLC.

In yet another embodiment, each of the compounds of Formula (II), Formula (III), and Formula (IV) is present in the composition in an amount in the range of about 0.001% to about 0.05% by weight, relative to the compound of Formula (I), when measured by HPLC.

In another embodiment, the present invention provides a composition comprising a triaminopyrimidine compound of Formula (I) having a purity of about 99.0% or more, when measured by area percentage of HPLC and a compound of Formula II, wherein the compound of Formula (II), is present, in an amount of about 0.15% or less by weight, relative to the compound of Formula (I), when measured by HPLC, for example in an amount of about 0.11% or less, about 0.1% or less, about 0.05% or less, or not in a detectable amount, by weight relative to the compound of Formula (I), when measured by HPLC.

In another embodiment, the present invention provides a composition comprising a triaminopyrimidine compound of Formula (I) having a purity of about 99.0% or more, when measured by area percentage of HPLC, and a compound Formula (II), wherein the compound of Formula (II) is present, in an amount of 0% to about 0.15% by weight, relative to the compound of Formula (I), when measured by HPLC.

In another embodiment, the compound of Formula (II) is present in the composition, in an amount of 0% to about 0.11% by weight, relative to the compound of Formula (I), when measured by HPLC.

In yet another embodiment, the compound of Formula (II) is present in the composition in an amount of about 0.001% to about 0.15% by weight, relative to the compound of Formula (I), when measured by HPLC.

In still yet another embodiment, the compound of Formula (II) is present in the composition in an amount of about 0.001% to about 0.11% by weight, relative to the compound of Formula (I), when measured by HPLC.

In still yet another embodiment, the compound of Formula (II) is present in the composition in an amount of about 0.01% to about 0.15% by weight, relative to the compound of Formula (I), when measured by HPLC.

In still yet another embodiment, the compound of Formula (II) is present in the composition in an amount of about 0.01% to about 0.11% by weight, relative to the compound of Formula (I), when measured by HPLC.

In still yet another embodiment, the compound of Formula (II) is present in the composition in an amount of about 0.001% to about 0.05% by weight, relative to the compound of Formula (I), when measured by HPLC.

In another embodiment, the present invention provides a composition comprising a triaminopyrimidine compound of Formula (I) having a purity of about 99.0% or more, when measured by area percentage of HPLC and a compound of Formula (III), wherein the compound of Formula (III), is present, in an amount of about 0.15% or less by weight, relative to the compound of Formula (I), when measured by HPLC, for example in an amount of about 0.11% or less, about 0.1% or less, about 0.05% or less, or not in a detectable amount, by weight relative to the compound of Formula (I), when measured by HPLC.

In another embodiment, the present invention provides a composition comprising a triaminopyrimidine compound of Formula (I) having a purity of about 99.0% or more, when measured by area percentage of HPLC, and a compound Formula (III), wherein the compound of Formula (III) is present, in an amount of 0% to about 0.15% by weight, relative to the compound of Formula (I), when measured by HPLC.

In another embodiment, the compound of Formula (III) is present in the composition, in an amount of 0% to about 0.11% by weight, relative to the compound of Formula (I), when measured by HPLC.

In yet another embodiment, the compound of Formula (III) is present in the composition in an amount of about 0.001% to about 0.15% by weight, relative to the compound of Formula (I), when measured by HPLC.

In still yet another embodiment, the compound of Formula (III) is present in the composition in an amount of about 0.001% to about 0.11% by weight, relative to the compound of Formula (I), when measured by HPLC.

In still yet another embodiment, the compound of Formula (III) is present in the composition in an amount of about 0.01% to about 0.15% by weight, relative to the compound of Formula (I), when measured by HPLC.

In still yet another embodiment, the compound of Formula (III) is present in the composition in an amount of about 0.01% to about 0.11% by weight, relative to the compound of Formula (I), when measured by HPLC.

In still yet another embodiment, the compound of Formula (III) is present in the composition in an amount of about 0.001% to about 0.05% by weight, relative to the compound of Formula (I), when measured by HPLC.

In another embodiment, the present invention provides composition comprising a triaminopyrimidine compound of Formula (I) having a purity of about 99.0% or more, when measured by area percentage of HPLC and a compound of Formula (IV), wherein the compound of Formula (IV), is present, in an amount of about 0.15% or less by weight, relative to the compound of Formula (I), when measured by HPLC, for example in an amount of about 0.11% or less, about 0.1% or less, about 0.05% or less, or not in a detectable amount, by weight relative to the compound of Formula (I), when measured by HPLC.

In another embodiment, the present invention provides a composition comprising a triaminopyrimidine compound of Formula (I) having a purity of about 99.0% or more, when measured by area percentage of HPLC, and a compound Formula (IV), wherein the compound of Formula (IV) is present, in an amount of 0% to about 0.15% by weight, relative to the compound of Formula (I), when measured by HPLC.

In another embodiment, the compound of Formula (IV) is present in the composition, in an amount of 0% to about 0.11% by weight, relative to the compound of Formula (I), when measured by HPLC.

In yet another embodiment, the compound of Formula (IV) is present in the composition in an amount of about 0.001% to about 0.15% by weight, relative to the compound of Formula (I), when measured by HPLC.

In still yet another embodiment, the compound of Formula (IV) is present in the composition in an amount of about 0.001% to about 0.11% by weight, relative to the compound of Formula (I), when measured by HPLC.

In still yet another embodiment, the compound of Formula (IV) is present in the composition in an amount of about 0.01% to about 0.15% by weight, relative to the compound of Formula (I), when measured by HPLC.

In still yet another embodiment, the compound of Formula (IV) is present in the composition in an amount of about 0.01% to about 0.11% by weight, relative to the compound of Formula (I), when measured by HPLC.

In still yet another embodiment, the compound of Formula (IV) is present in the composition in an amount of about 0.001% to about 0.05% by weight, relative to the compound of Formula (I), when measured by HPLC.

In another embodiment, the present invention provides a composition comprising a triaminopyrimidine compound of Formula (I) and one or more of compounds of Formula (II), Formula (III), and Formula (IV).

In another general aspect, the present invention provides a composition comprising a triaminopyrimidine compound of Formula (I) having a purity of about 99.0% or more, when measured by area percentage of HPLC and one or more compounds of Formula (V), or Formula (VI), each present, in an amount of about 500 ppm or less, when measured by HPLC.

In another embodiment, the present invention provides a composition comprising a triaminopyrimidine compound of Formula (I) having a purity of about 99.0% or more, when measured by area percentage of HPLC, and one or more compounds of Formula (V), or Formula (VI), wherein each of the compounds of Formula (V), or Formula (VI) is present in an amount of about 134 ppm or less when measured by HPLC.

In another embodiment, the present invention provides a composition comprising a triaminopyrimidine compound of Formula (I) having a purity of about 99.0% or more, when measured by area percentage of HPLC and one or more compounds of Formula (V), or Formula (VI), each present, in an amount of upto 500 ppm, when measured by HPLC.

In another embodiment, the present invention provides a composition comprising a triaminopyrimidine compound of Formula (I) having a purity of about 99.0% or more, when measured by area percentage of HPLC and one or more compounds of Formula (V), or Formula (VI), each present, in an amount of upto 134 ppm, when measured by HPLC.

In another embodiment, the present invention provides a composition comprising a triaminopyrimidine compound of Formula (I) having a purity of about 99.0% or more, when measured by area percentage of HPLC and one or more compounds of Formula (V), or Formula (VI), each present, in an amount in the range of about 0 ppm to about 500 ppm, when measured by HPLC.

In another embodiment, the present invention provides a composition comprising a triaminopyrimidine compound of Formula (I) having a purity of about 99.0% or more, when measured by area percentage of HPLC and one or more compounds of Formula (V), or Formula (VI), each present, in an amount in the range of about 0 ppm to about 134 ppm, when measured by HPLC.

In another embodiment, each of the compounds of Formula (V) or Formula (VI) is present in the composition in an amount of about 1.0 ppm to about 500 ppm, when measured by HPLC.

In another embodiment, each of the compounds of Formula (V) or Formula (VI) is present in the composition in an amount of about 2.0 ppm to about 500 ppm, when measured by HPLC.

In another embodiment, each of the compounds of Formula (V) or Formula (VI) is present in the composition in an amount of about 1.0 ppm to about 134 ppm, when measured by HPLC.

In another embodiment, each of the compounds of Formula (V) or Formula (VI) is present in the composition in an amount of about 2.0 ppm to about 134 ppm, when measured by HPLC.

In another embodiment, each of the compounds of Formula (V) or Formula (VI) is present in the composition in an amount of about 4.0 ppm to about 134 ppm, when measured by HPLC.

In another embodiment, each of the compounds of Formula (V), or Formula (VI), when present in the composition, is in a detectable amount of about 134 ppm or less, when measured by HPLC.

In another embodiment, the present invention provides a composition comprising a triaminopyrimidine compound of Formula (I) having a purity of about 99.0% or more, when measured by area percentage of HPLC and a compound of Formula (V), wherein the compound of Formula (V) is present, in an amount of 500 ppm or less, when measured by HPLC, for example in an amount of about 400 ppm or less, about 300 ppm or less, about 200 ppm or less, about 150 ppm or less, about 134 ppm or less, about 100 ppm or less, about 50 ppm or less, or not in a detectable amount, when measured by HPLC.

In another embodiment, the compound of Formula (V) is present in the composition, in an amount of 134 ppm or less, when measured by HPLC.

In another embodiment, the compound of Formula (V) is present in the composition in an amount of upto 500 ppm, when measured by HPLC.

In yet another embodiment, the compound of Formula (V) is present in the composition in an amount of upto 134 ppm, when measured by HPLC.

In yet another embodiment, the compound of Formula (V) is present in the composition in an amount in the range of 0 ppm to about 500 ppm, when measured by HPLC.

In yet another embodiment, the compound of Formula (V) is present in the composition in an amount in the range of about 0 ppm to about 134 ppm, when measured by HPLC.

In yet another embodiment, the compound of Formula (V) is present in the composition in an amount of in the range of about 1 ppm to about 134 ppm, when measured by HPLC.

In yet another embodiment, the compound of Formula (V) is present in the composition in an amount of in the range of about 4 ppm to about 134 ppm, when measured by HPLC.

In yet another embodiment, the compound of Formula (V) is present in the composition in an amount of in the range of about 1 ppm to about 100 ppm, when measured by HPLC.

In another embodiment, the present invention provides a composition comprising a triaminopyrimidine compound of Formula (I) having a purity of about 99.0% or more, when measured by area percentage of HPLC and a compound of Formula (VI), wherein the compound of Formula (VI), is present, in an amount of 500 ppm or less, when measured by HPLC, for example in an amount of about 400 ppm or less, about 300 ppm or less, about 200 ppm or less, about 150 ppm or less, about 134 ppm or less, about 100 ppm or less, about 50 ppm or less, or not in a detectable amount, when measured by HPLC.

In another embodiment, the compound of Formula (VI), is present in the composition in an amount of about 134 ppm or less, when measured by HPLC.

In another embodiment, the compound of Formula (VI) is present in the composition in an amount of upto 500 ppm, when measured by HPLC.

In yet another embodiment, the compound of Formula (VI) is present in the composition in an amount of upto 134 ppm, when measured by HPLC.

In yet another embodiment, the compound of Formula (VI) is present in the composition in an amount in the range of about 0 ppm to about 500 ppm, when measured by HPLC.

In yet another embodiment, the compound of Formula (VI) is present in the composition in an amount in the range of about 0 ppm to about 134 ppm, when measured by HPLC.

In yet another embodiment, the compound of Formula (VI) is present in the composition in an amount of in the range of about 1 ppm to about 134 ppm, when measured by HPLC.

In yet another embodiment, the compound of Formula (VI) is present in the composition in an amount of in the range of about 4 ppm to about 134 ppm, when measured by HPLC.

In yet another embodiment, the compound of Formula (VI) is present in the composition in an amount in the range of about 1 ppm to about 100 ppm, when measured by HPLC.

In another embodiment, the present invention provides a composition comprising a triaminopyrimidine compound of Formula (I) having a purity of about 99.0% or more, when measured by area percentage of HPLC, a compound of Formula (V) and a compound of Formula (VI), wherein each of the compound of the compound of Formula (V) and Formula (VI) is present in an amount of about 0 ppm to about 500 ppm, when measured by HPLC.

In yet another embodiment, the present invention provides a composition comprising a triaminopyrimidine compound of Formula (I) having a purity of about 99.0% or more, when measured by area percentage of HPLC, a compound of Formula (V) and a compound of Formula (VI), wherein each of the compound of the compound of Formula (V) and Formula (VI) is present in an amount of about 0 ppm to about 134 ppm, when measured by HPLC.

In another embodiment, the present invention provides a composition comprising a triaminopyrimidine compound of Formula (I), and one or more compounds of Formula (V) or Formula (VI), wherein each of the compounds of Formula (V) or Formula (V) is present in a non-detectable amount, when measured by HPLC.

The composition of the present invention is found to be stable, when stored for at least 6 months at 40° C.±2° C. temperature and 75%+5% relative humidity, or at 25° C.±2° C. temperature and 60±5% relative humidity, wherein after storage, each of the compounds of Formula (II), Formula (III), or Formula (IV), is present, in an amount of about 0.15%, or less, by weight, relative to the compound of Formula (I), when measured by HPLC, and each of the compounds of Formula (V), or Formula (V) is present in an amount of about 500 ppm or less, when measured by HPLC.

In another embodiment, each of the compounds of Formula (II), Formula (III), or Formula (IV), is present, in an amount of about 0.11%, or less, by weight, relative to the compound of Formula (I), when measured by HPLC, and each of the compounds of Formula (V), or Formula (V) is present in an amount of about 134 ppm or less, when measured by HPLC, when the composition is stored for at least 6 months at 40° C.±2° C. temperature and 75%+5% relative humidity, or at 25° C.±2° C. temperature and 60%+5% relative humidity.

In another embodiment, the present invention provides a stable composition comprising a triaminopyrimidine compound of Formula (I), having a purity of about 99.0% or more, when measured by area percentage of HPLC, and one or more of compounds of Formula (II), Formula (III), Formula (IV), Formula (V), and Formula (VI), wherein each of the compounds of Formula (II), Formula (III), or Formula (IV), is present, in an amount of about 0.15%, or less, by weight, relative to the compound of Formula (I), when measured by HPLC, and each of the compounds of Formula (V), or Formula (V) is present in an amount of about 134 ppm or less, when measured by HPLC, when the composition is stored for at least 6 months at 40° C.±2° C. temperature and 75%+5% relative humidity, or at 25° C.±2° C. temperature and 60%+5% relative humidity.

In yet another embodiment, each of the compounds of Formula (II), Formula (III), or Formula (IV), is present in the composition, in an amount of about 0.11%, or less, by weight, relative to the compound of Formula (I), when measured by HPLC.

In another general aspect, the present invention provides a triaminopyrimidine compound of Formula (I), having a purity of about 99% or more, as determined by area percentage of HPLC and containing:
(i) a compound of Formula (II), in an amount of about 0.15% or less,
(ii) a compound of Formula (III), in an amount of about 0.15% or less,
(iii) a compound of Formula (IV), in an amount of about 0.15% or less, by weight relative to the compound of Formula (I), when measured by HPLC; or
(iv) a compound of Formula (V) in an amount of about 500 ppm or less, or
(v) a compound of Formula (VI), in an amount of about 500 ppm or less, when measured by HPLC.

In another embodiment, the compound of Formula (I) of the present invention is having a purity of about 99% or more as determined by area percentage of HPLC, for example, a purity of about 99.5% or more, about 99.8% or more, as determined by area percentage of HPLC.

In another embodiment, the triaminopyrimidine compound of Formula (I) of the present invention contains the compound of Formula (II), in an amount of about 0.15% or less, for example, in an amount of about 0.11% or less, about 0.1% or less, about 0.05% or less, or not in a detectable amount, by weight relative to the compound of Formula (I), when measured by HPLC.

In another embodiment, the triaminopyrimidine compound of Formula (I) of the present invention contains the compound of Formula (III), in an amount of about 0.15% or less, for example, in an amount of about 0.11% or less, about 0.1% or less, about 0.05% or less, or not in a detectable amount, by weight relative to the compound of Formula (I), when measured by HPLC.

In another embodiment, the triaminopyrimidine compound of Formula (I) of the present invention contains the compound of Formula (IV), in an amount of about 0.15% or less, for example, in an amount of about 0.11% or less, about 0.1% or less, about 0.05% or less, or not in a detectable amount, by weight relative to the compound of Formula (I), when measured by HPLC.

In another embodiment, the triaminopyrimidine compound of Formula (I) of the present invention contains the compound of Formula (V) in an amount of about 500 ppm or less, for example in an amount of about 400 ppm or less, about 300 ppm or less, about 200 ppm or less, about 150 ppm or less, about 134 ppm or less, about 100 ppm or less, about 50 pm or less, or not in a detectable amount, when measured by HPLC.

In another embodiment, the triaminopyrimidine compound of Formula (I) of the present invention contains the compound of Formula (V) in an amount of about 134 ppm or less when measured by HPLC.

In another embodiment, the triaminopyrimidine compound of Formula (I) of the present invention contains the compound of Formula (VI) in an amount of about 500 ppm or less, for example in an amount of about 400 ppm or less, about 300 ppm or less, about 200 ppm or less, about 150 ppm or less, about 134 ppm or less, about 100 ppm or less, about 50 pm or less, or not in a detectable amount, when measured by HPLC.

In another embodiment, the triaminopyrimidine compound of Formula (I) of the present invention contains the compound of Formula (VI) in an amount of about 134 ppm or less when measured by HPLC.

In another general aspect, the present invention provides a process for the preparation of a compound of Formula (I), having a purity of about 99.0% or more, when measured by area percentage of HPLC, and substantially free of one or more compounds of Formula (II), Formula (III), Formula (IV), Formula (V), or Formula (VI), the process comprising:
(a) reacting a compound of Formula (X),

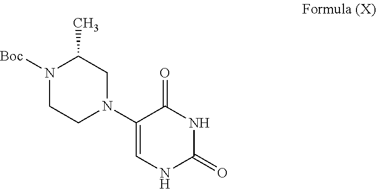 [SMILES: C[C@@H]1CN(c2c[nH]c(=O)[nH]c2=O)CCN1C(=O)OC(C)(C)C]

with phosphorus oxychloride at a temperature of about 100° C. to about 115° C., to obtain a compound of Formula (IX), or a salt thereof,

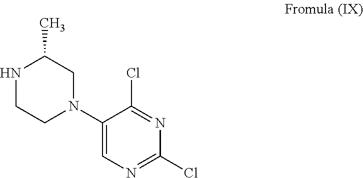

(b) reacting the compound of Formula (IX), or a salt thereof, with a methylating agent, in the presence of a reducing agent, in one or more solvents at a temperature of about 10° C. to about 30° C., to obtain a compound of Formula (VII), or a salt thereof,

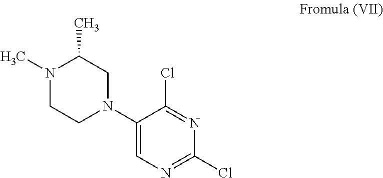 [SMILES: C[C@@H]1CN(c2cnc(Cl)nc2Cl)CCN1C]

(c) reacting the compound of Formula (VII), or a salt thereof, with a compound of Formula (VI), or a salt thereof, in the presence of one or more bases in one or more solvents, at a temperature of about 90° C. to about 120° C.

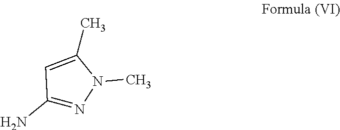

to obtain a compound of Formula (VIII), or a salt thereof.

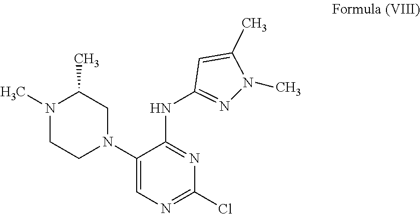

(d) reacting the compound of Formula (VIII), or a salt thereof, with a compound of Formula (V), or a salt, or a hydrate thereof, in the presence of a Grignard reagent used in an amount of about 3 to about 6 mole equivalents relative to compound of Formula (VIII), in one or more solvents,

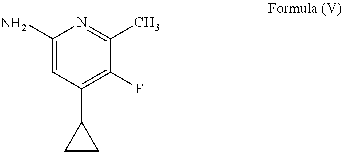

to obtain the compound of Formula I, and
(e) crystallizing the compound of Formula (I) in one or more solvents to obtain the compound of Formula (I) having a purity of about 99.0% or more, when measured by area percentage of HPLC and substantially free of one or more compounds of Formula (II), Formula (III), Formula (IV), Formula (V), or Formula (VI).

In general, the compound of Formula (IX), or a salt thereof and the compound of Formula (VII), or a salt thereof is not isolated. i.e. these are formed in-situ and converted into next-step without isolating.

In general, the methylating agent at step (b) is selected from diazomethane, 2,2-dimethoxypropane, dimethyl carbonate, dimethyl sulfide, dimethyl zinc, methyl fluorosulfonate, methyl iodide, methyl bromide, methyl trifluoromethanesulfonate, trimethoxonium tetrafluoroborate, formaldehyde, and a mixture of formic acid and formaldehyde. In particular, the methylating agent is formaldehyde.

In another embodiment, the methylating agent at step (b) is formaldehyde.

In general, the reducing agent at step (b) is selected from sodium triacetoxyborohydride, sodium borohydride, and sodium cyanoborohydride. In particular, the reducing agent used at step (b) is sodium triacetoxyborohydride.

In general, the compound of (X) is reacted with POCl₃ in the presence of catalytic amount of pyridine to obtain the intermediate compound of Formula (IX), which is not isolated and reacted with formaldehyde in the presence sodium triacetoxyborohydride to obtain the compound of Formula (VII). The reaction mixture may be basified and extracted with one or more solvents to obtain the compound of Formula (VII). The compound of Formula (VII) may be obtained by removal of solvent after extraction. The reaction mixture may be basified using one or more bases selected from sodium hydroxide, potassium hydroxide, lithium hydroxide, barium hydroxide, sodium carbonate, potassium carbonate, barium carbonate, cesium carbonate, sodium bicarbonate, and potassium bicarbonate. In particular, sodium carbonate may be used.

In general, the one or more bases at step (c) are selected from diisopropylethylamine, triethylamine, and 1,8-diazabicyclo(5.4.0) undec-7-ene. In particular, the base is diisopropylethylamine.

In general, the one or more solvents at step (c) are selected from toluene, tetrahydrofuran, 1,4-dioxane, xylene, dimethylsulfoxide, and dimethylacetamide. In particular, the solvent is toluene.

In general, the Grignard reagent at step (d) is selected from isopropyl magnesium chloride, cyclohexyl magnesium chloride, tert-butyl magnesium chloride, p-tolyl magnesium chloride, hexyl magnesium chloride, isopentyl magnesium chloride, cyclohexyl magnesium chloride, and cyclohexyl magnesium bromide. In particular, the Grignard reagent is selected from isopropyl magnesium chloride or tert-butyl magnesium chloride, more particularly isopropyl magnesium chloride.

In general, the solvent at step (d) is selected from one or more of tetrahydrofuran, 2-methyl tetrahydrofuran, methyl tert-butyl ether, di-isopropyl ether, toluene, and 1,4-dioxane. In particular, the solvent is toluene or tetrahydrofuran. More particularly, the solvent is a mixture of toluene and tetrahydrofuran.

In another embodiment, the Grignard reagent at step (d) is used in an amount of about 3 to about 6 mole equivalents relative to the compound of Formula (VIII). In particular, the Grignard reagent is used in an amount of about 4.5 to about 5.5 mole equivalents relative to the compound of Formula (VIII), more particularly, in an amount of about 5.5 mole equivalents relative to the compound of Formula (VIII).

In general, the reaction at step (d) is performed at a temperature of about 10° C. to about 40° C. In particular, the reaction at step (d) performed at a temperature of about 20° C. to about 30° C.

In general, the reaction at step (d) may be carried out for a time sufficient for the completion of reaction, for example 1-4 hours. After completion, the reaction mixture can be quenched with an addition of an acid such as dil. HCl and then basified to a pH of 8 to 10 by the addition of aqueous base such as sodium carbonate. The product then can be extracted with a suitable solvent and concentrated to obtain the compound of Formula (I).

In general, the compound of Formula (V) used at step (d) is in the form of monohydrochloride monohydrate.

In general, the solvents at step (e) is selected from one or more of water, methanol, acetone, acetonitrile, dichloromethane, and ethyl acetate. In particular, the solvent is ethyl acetate.

In another embodiment, the one or more solvents at step (e) is used in an amount of 10 volumes to 20 volumes of the quantity of the compound of Formula (I), particularly, 10 volumes to 15 volumes of the quantity of the compound of Formula (I).

In another general aspect, the present invention provides a pharmaceutical composition comprising a triaminopyrimidine compound of Formula (I) having a purity of about 99.0% or more, when measured by area percentage of HPLC, and substantially free of one or more compounds of Formula (II), Formula (III), Formula (IV), Formula (V) or Formula (VI), together with one or more pharmaceutically acceptable excipients.

In another general aspect, the present invention provides a pharmaceutical composition comprising a triaminopyrimidine compound of Formula (I) and a compound of Formula (II), together with one or more pharmaceutically acceptable excipients.

In another general aspect, the present invention provides a pharmaceutical composition comprising a triaminopyrimidine compound of Formula (I) and a compound of Formula (III), together with one or more pharmaceutically acceptable excipients.

In another general aspect, the present invention provides a pharmaceutical composition comprising a triaminopyrimidine compound of Formula (I) and a compound of Formula (IV), together with one or more pharmaceutically acceptable excipients.

In another general aspect, the present invention provides a pharmaceutical composition comprising a triaminopyrimidine compound of Formula (I) having a purity of about 99.0% or more, when measured by area percentage of HPLC and one or more of compounds of Formula (II), Formula (III), Formula (IV), Formula (V), or Formula (VI), wherein each of the compounds of Formula (II), Formula (III), or Formula (IV) is present, in an amount of about 0.15%, or less, by weight, relative to the triaminopyrimidine compound of Formula (I), when measured by HPLC, and each of the compounds of Formula (V), or Formula (VI) is present in an amount of about 134 ppm or less, when measured by HPLC, together with one or more pharmaceutically acceptable excipients.

In another embodiment, each of the compounds of Formula (II), Formula (III), or Formula (IV) is present in the pharmaceutical composition, in an amount of about 0.11%, or less, by weight, relative to the triaminopyrimidine compound of Formula (I), when measured by HPLC.

### Abbreviations

HPLC: High performance liquid chromatography
LOD: Limit of detection
LOQ: Limit of quantification
BQL: Below Quantification Limit
RH: Relative Humidity

### HPLC Method of Analysis:

HPLC purity of compound of Formula (I) and the content of compounds of Formula (II), Formula (III), or Formula (IV), were measured by HPLC using following conditions: Equipment: HPLC system or equivalent, Column: C 18, 5 μm (250 mm×4.6 mm), Detector: UV-VIS detector or PDA detector, Flow rate: 1.2 mL/min, Wavelength: 265 nm, Injection Vol.: 10 μL, Column Oven Temp.: 50° C., Run Time: 80 minutes.

### Gradient Composition:

| | % Mobile phase B relative to Mobile phase |
|---|---|
| Time (minutes) | A |
| 0 | 30 |
| 50 | 100 |
| 70 | 100 |
| 72 | 30 |
| 80 | 30 |

Preparation of Buffer solution: 10 mM of Dipotassium hydrogen phosphate solution was prepared and adjusted to the pH 6.8±0.05 with 10% Orthophosphoric acid solution.

Preparation of Mobile phase-A: A mixture of Buffer solution: Acetonitrile in the volume ratio of 95:5.

Preparation of Mobile phase-B: A mixture of Acetonitrile: Methanol: Milli Q water in the volume ratio of 60:20:20.

Preparation of Diluent: A mixture of Milli Q water: Acetonitrile in the volume ratio of 40:60.

Preparation of sample solution: 1000 ppm of compound of Formula (I) in diluent.

TABLE 1
Summary of HPLC analysis of triaminopyrimidine
compound of Formula (I) along with compound of
Formula (II), Formula (III) and Formula (IV)
Compound of Formula (I): HPLC purity: 99.7%

| Impurities | Results | LOD | LOQ |
|---|---|---|---|
| Formula (II) | ND | 0.01 | 0.04 |
| (% w/w) | | | |
| Formula (III) | 0.02 | 0.01 | 0.04 |
| (% w/w) | | | |
| Formula (IV) | 0.01 | 0.01 | 0.04 |
| (% w/w) | | | |

N.D. indicates “Not Detected”

The contents of compounds of Formula (V) and Formula (VI) were measured by HPLC using following conditions:

Equipment: HPLC system or equivalent, Column: C18, 5 μm (250 mm×4.6 mm), Detector: UV-VIS detector or PDA detector, Flow rate: 1.0 mL/min, Wavelength: 220 nm, Injection Vol.: 10 μL, Column Oven Temp.: 40° C., Run Time: 75 minutes.

### Gradient Composition:

| | % Mobile phase B relative to Mobile |
|---|---|
| Time (minutes) | phase A |
| 0 | 5 |
| 45 | 50 |
| 47 | 100 |
| 65 | 100 |
| 67 | 5 |
| 75 | 5 |

Preparation of Buffer solution: 10 mM of Dipotassium hydrogen phosphate solution was prepared and adjusted to the pH 6.5±0.05 with 10% Orthophosphoric acid solution.

Preparation of Mobile phase A: A mixture of Buffer solution: Methanol in the volume ratio of 95:5.

Preparation of Mobile phase B: A mixture of Acetonitrile: Milli Q water in the volume ratio of 80:20.

Preparation of diluent: Methanol as a diluent.

Preparation of sample solution: 5 mL of Diluent was added to about 200 mg of compound of Formula (I) and sonicated to dissolve. Further, 5 mL of Milli Q water was added and sonicated.

TABLE 2
Summary of HPLC analysis of triaminopyrimidine compound of
Formula (I) along with compound of Formula (V) and Formula (VI)
Compound of Formula (I): HPLC purity: 99.7%

| Impurities | Results | LOD | LOQ |
|---|---|---|---|
| Formula (V) | 13.62 | 4.41 | 13.37 |
| (ppm) | | | |
| Formula (VI) | ND | 4.48 | 13.56 |
| (ppm) | | | |

N.D. indicates “Not Detected”

TABLE 3
Stability data at 40° C. ± 2° C. (75% ± 5% RH)

| Sr. | | | 1 | 2 | 3 | 6 |
|---|---|---|---|---|---|---|
| No | Tests | Initial | Month | months | months | months |
| 1 | Compound of | 99.7% | 99.7% | 99.7% | 99.7% | 99.76% |
| | Formula (I) | | | | | |
| | HPLC purity | | | | | |

Related substance by HPLC (% w/w)

| | | | | | | |
|---|---|---|---|---|---|---|
| 2 | Formula (II) | ND | ND | ND | ND | ND |
| 3 | Formula (III) | 0.02 | 0.03 | 0.02 | 0.02 | BQL |
| 4 | Formula (IV) | 0.01 | 0.01 | 0.01 | 0.004 | BQL |

Content by HPLC (ppm)

| | | | | | | |
|---|---|---|---|---|---|---|
| 6 | Formula (V) | 13.62 | — | — | 19.74 | 18.26 |
| 7 | Formula (VI) | ND | — | — | ND | ND |

N.D. indicates “Not Detected”
BQL—indicates “Below quantification limit”

TABLE 4
Stability data at 25° C. ± 2° C. (60% ± 5% RH)

| Sr. No | Tests | Initial | 3 Months | 6 Months |
|---|---|---|---|---|
| 1 | Compound of Formula (I) | 99.7% | 99.7% | 99.76% |
| | HPLC purity | | | |

Related substance by HPLC (% w/w)

| | | | | |
|---|---|---|---|---|
| 2 | Formula (II) | ND | ND | ND |
| 3 | Formula (III) | 0.02 | 0.02 | BQL |
| 4 | Formula (IV) | 0.01 | 0.003 | BQL |

Content by HPLC (ppm)

| | | | | |
|---|---|---|---|---|
| 6 | Formula (V) | 13.62 | 18.99 | 16.61 |
| 7 | Formula (VI) | ND | ND | ND |

N.D. indicates “Not Detected”
BQL-indicates “Below quantification limit”

The present invention is further illustrated by the following examples which is provided merely to be exemplary of the invention and do not limit the scope of the invention. Certain modifications and equivalents will be apparent to those skilled in the art and are intended to be included within the scope of the present invention. The examples are set forth to aid in understanding the invention but are not intended to, and should not be construed to limit its scope in any way. The examples do not include detailed descriptions of conventional methods. Such methods are well known to those of ordinary skill in the art and are described in various publications.

NMR spectra were recorded using Bruker Avance 400 MHz FT NMR spectrometer.

Mass was determined using mass spectrometer Qtrap-4500 from Applied Biosciences (ABSciex).

### EXAMPLES

### Example 1: Preparation of (R)-2,4-dichloro-5-(3,4-dimethylpiperazin-1-yl)pyrimidine (Compound of Formula (VII))

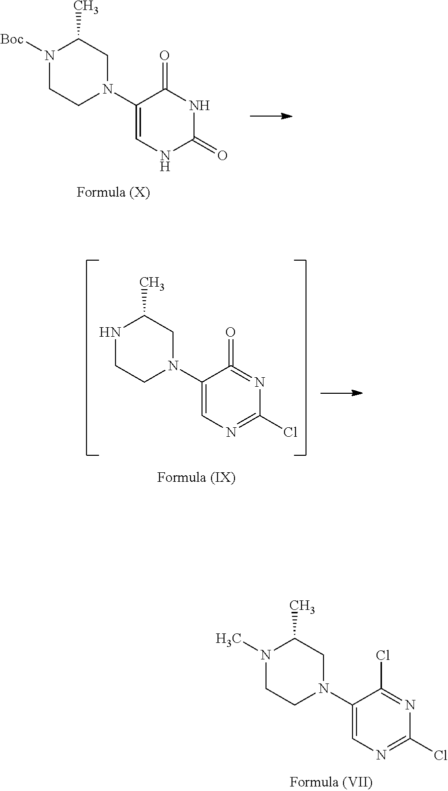

To a 250 ml 4 neck RBF, compound of Formula X (25 g, 0.08 mol), phosphorus oxychloride (5V) and pyridine (0.5 g, 0.0063 mol) were added. The temperature of the reaction was raised to 105° C. to 110° C. and stirred for 6H at 105° C. to 110° C. After completion of reaction, phosphorus oxychloride (˜3 to 3.5V) was distilled out and the reaction was cooled to 50° C. to 60° C. To this mixture, ethyl acetate (6V) was added and stirred, followed by distillation of ethyl acetate (˜3V) and the reaction mass was allowed to cool at 15° C. to 25° C. In another 1 liter 4 neck RBF water (3V) was added and cooled to 0° C. to 5° C. and the above reaction mass was quenched in water at 0° C. to 10° C. Further temperature of reaction mass was raised to 15° C. to 25° C. and the pH was adjusted to 6 to 7 by using 20% aqueous sodium carbonate solution at 15° C. to 25° C. To this reaction mass 13.1 g of 37% Aq. formaldehyde (13.1 g, 0.16 mol) was added and stirred for 10 minutes at 15° C. to 25° C. The reaction mass was cooled to 15° C. to 25° C. followed by addition of sodium triacetoxyborohydride (34.15 g, 0.16 mol) and stirred for 2 hours at this temperature. After completion of reaction, the pH was adjusted to 7 to 9 by using 20% aqueous sodium carbonate solution and stirred for 30 minutes. The layers were separated, and the product were back extracted from aqueous layer using ethyl acetate (5V). The product containing organic layer was distilled off to get crude desired product which was further used without purification in the next step.

1H NMR (400 MHz, CDCl₃) δ 8.19 (s, 1H), 3.37-3.25 (m, 2H), 3.06-2.88 (m, 2H), 2.68-2.46 (m, 2H), 2.39-2.34 (m, 4H), 1.13 (d, J=6.4 Hz, 3H). 13C NMR (100 MHz, CDCl₃) δ 156.1, 152.3, 149.9, 142.3, 57.3, 54.9, 50.5, 42.4, 16.7. ESIMS m/z: 261.1 [M⁺H]⁺ for C₁₀H₁₄C₁₂N₄.

### Example 2: Preparation of (R)-2-chloro-N-(1,5-dimethyl-1H-pyrazol-3-yl)-5-(3,4-dimethylpiperazin-1-yl)pyrimidin-4-amine (Compound of Formula (VIII))

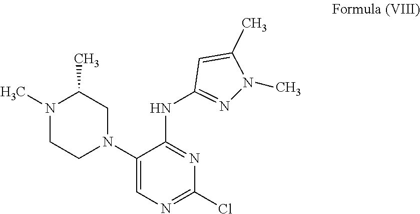

To a 250 ml 4 neck RBF, compound of Formula (VII), N,N-diisopropylethylamine (4V) and 1,5-dimethyl-1H-pyrazol-3-amine (DMPA) (compound of Formula (VI)) (11.16 g, 0.10 mol) were added at 25° C. to 35° C. The temperature of the reaction mass was raised to 95° C. to 105° C. and stirred for 16 hours. After completion of reaction, the reaction mixture was cooled to 60° C. to 80° C., followed by the addition of toluene (7V) and further cooled to 45° C. to 55° C. To this mixture, water (2V) was added and stirred for 30 minutes at 45° C. to 55° C. The layers were allowed to settle and the organic layer was separated. The aqueous layer was again extracted with Toluene (2V). The combined organic layer was distilled out to remove traces of solvent. The obtained residue was crystallized in acetonitrile (2V) and filtered off followed by drying to obtain pure compound of Formula (VIII) 15.0 g (Yield: 55%, Purity: 98.0%).

¹H NMR (400 MHz, DMSO-d6) δ 8.45 (s, 1H), 8.02 (d, J=0.8 Hz, 1H), 6.4 (s, 1H), 3.66 (s, 3H), 2.92-2.76 (m, 4H), 2.47-2.37 (m, 2H), 2.30-2.27 (m, 4H), 2.22 (s, 3H), 0.99 (d, J=6 Hz, 3H). 13C NMR (100 MHz, DMSO-d6) δ 156.3, 154.3, 147.6, 144.7, 139.5, 131.2, 97.9, 57.5, 58.0, 55.3, 51.4, 42.6, 35.9, 17.0, 11.3. ESIMS m/z: 336.2 [M+H]⁺ for C₁₅H₂₂ClN₇.

### Example 3: Preparation of (R)—N²-(4-cyclopropyl-5-fluoro-6-methylpyridin-2-yl)-N⁴-(1,5-dimethyl-1H-pyrazol-3-yl)-5-(3,4-dimethylpiperazin-1-yl)pyrimidine-2,4-diamine (Compound of Formula (I))

To a 250 ml 4 neck round bottom flask, a compound of Formula (VIII) (5 g, 0.015 mol), 4-cyclopropyl-5-fluoro-6-methylpyridin-2-amine hydrochloride monohydrate (compound of Formula (V). HCl·H₂O; 3.45 g, 0.016 mol) and toluene (5V) at 25° C. to 35° C. were added. To this mixture 2M solution of isopropylmagnesium chloride (iPrMgCl) in THF (41 ml, 0.083 mol) were added at 20° C. to 30° C. The resulting reaction mixture was agitated for a minimum of 4 hours. The resulting slurry was quenched into previously cooled dil. HCl sol (15 ml Con. HCl+15 ml Water). at 15 to 35° C. and flushed RBF with toluene (3V) and stirred for 30 minutes. To this 20% aqueous solution of sodium carbonate was added and pH adjusted to 8 to 10 The temperature of reaction mass was raised to 40° C. to 50° C. and stirred for 1 hour. The obtained slurry was filtered through celite bed and filtrate were collected. The layers were separated, and the aqueous layer was again extracted with using toluene (5V). The combined organic layer was distilled out under vacuum to obtain the crude material. Crystallization of this crude material in ethyl acetate (12V) followed by vacuum drying afforded the compound of Formula I, 4.0 g (Yield: 73%, Purity: 99.7%).

### Structure Characterization of Impurities:

| Impurity | Characterization |
|---|---|
| Compound of | ¹H NMR (400 MHz, DMSO-d₆) δ 9.24 (s, 1H), 8.27 (s, 1H), 8.04 (s, |
| Formula (II) | 1H), 7.78 (d, J = 4.4 Hz, 1H), 6.26 (s, 1H), 3.63 (s, 3H), 2.90-2.76 (m, |
| | 4H), 2.57-2.51 (m, 1H), 2.35-2.34 (m, 4H), 2.26-2.22 (m, 7H), 2.09-2.05 |
| | (m, 1H), 1.05-1.01 (m, 5H), 0.88-0.87 (m, 2H). |
| | ¹³C NMR (100 MHz, DMSO-d₆) δ 156.02, 155.64, 154.58, 152.14, |
| | 148.50, 147.50, 147.24, 147.21, 143.31, 143.11, 141.44, 141.30, |
| | 139.01, 124.65, 107.78, 96.74, 59.64, 56.29, 52.63, 58.24, 42.43, |
| | 35.51, 17.53, 11.48, 9.22, 9.17, 9.14, 9.11. ESIMS m/z: calculated for |
| | C₂₄H₃₂FN₉: 465.28, found: 466.0 [M + H]⁺. |
| Compound of | ¹H NMR (400 MHz, CDCl₃) δ 8.11 (s, 1H), 8.04 (s, 1H), 7.71 (s, 1H), |
| Formula (III) | 7.65 (d, J = 4.8 Hz, 1H), 6.55 (s, 1H), 3.80 (s, 1H), 3.66 (s, 3H), |
| | 3.62-3.39 (m, 4H), 3.17 (s, 3H), 2.80-2.62 (m, 2H), 2.38 (d, J = 4.8 Hz, 3H), |
| | 2.24 (s, 3H), 2.09-2.02 (m, 1H), 1.30 (d, J = 4.8 Hz, 3H), 1.03-0.98 |
| | (m, 2H), 0.85-0.81 (m, 2H). |
| | ¹³C NMR (100 MHz, CDCl₃) δ 155.93, 155.45, 154.15, 151.72, |
| | 148.50, 148.15, 148.11, 146.10, 142.86, 142.67, 141.50, 141.36, |
| | 139.55, 123.97, 106.39, 97.25, 69.09, 68.11, 57.24, 53.32, 46.82, |
| | 35.54, 17.46, 11.79, 11.42, 9.24, 9.19, 8.99. ESIMS m/z: calculated for |
| | C₂₄H₃₂FN₉O: 481.27, found: 482.2 [M + H]⁺. |
| Compound of | ¹H NMR (400 MHz, CDCl₃) δ 7.95 (s, 1H), 7.93 (s, 1H), 7.72 (d, J = |
| Formula (IV) | 4.8 Hz, 1H), 7.60 (s, 1H), 6.59 (s, 1H), 4.91-4.55 (m, 1H), 3.72 (s, 3H), |
| | 3.70-3.50 (m, 1H), 3.11-2.86 (m, 5H), 2.42 (d, J = 2.8 Hz, 3H), 2.30 |
| | (s, 3H), 2.17-2.02 (m, 4H), 1.56-1.44 (m, 3H), 1.07-1.02 (m, 2H), |
| | 0.89-0.85 (m, 2H). |
| | ¹³C NMR (100 MHz, CDCl₃) δ 169.11, 155.69, 155.55, 154.11, |
| | 151.68, 148.25, 148.22, 147.03, 145.88, 142.80, 142.61, 141.46, |
| | 141.32, 139.42, 124.82, 106.37, 97.43, 49.91, 44.53, 57.35, 57.12, |
| | 52.58, 52.40, 42.27, 36.99, 35.56, 21.77, 21.15, 17.51, 16.54, 15.45, |
| | 11.44, 9.24, 9.19, 8.95. ESIMS m/z: calculated for C₂₅H₃₂FN₉O: |
| | 493.27, found: 494.3 [M + H]⁺. |

While the present invention has been described in terms of its specific embodiments, certain modifications and equivalents will be apparent to those skilled in the art and are intended to be included within the scope of the present invention.

1. A compound selected from a compound of Formula (II), a compound of Formula (III), or a compound of Formula (IV),

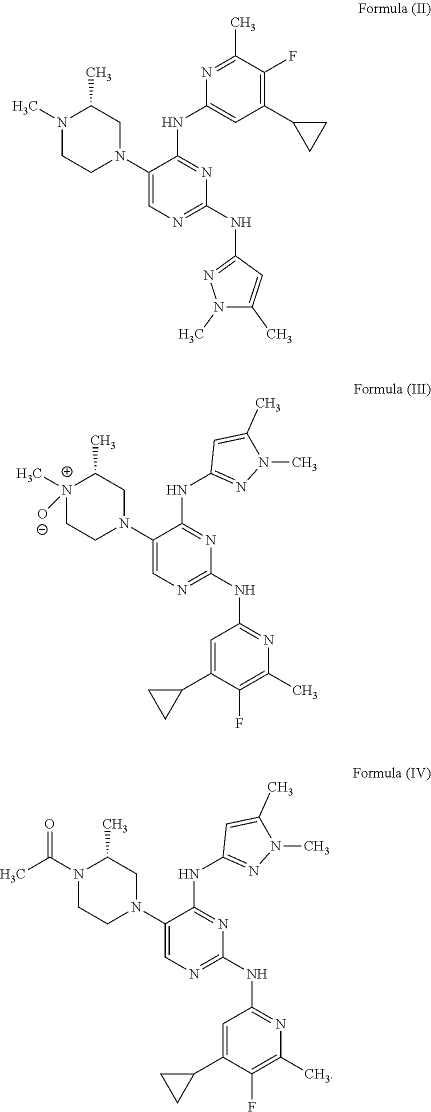

2. A composition comprising a triaminopyrimidine compound of Formula (I) 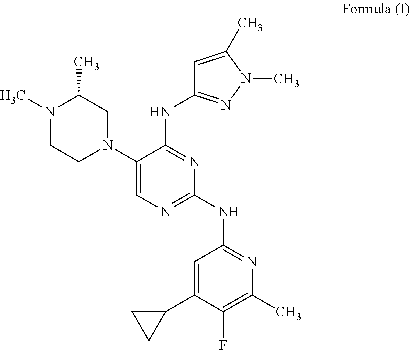
having a purity of about 99.0% or more, when measured by area percentage of high performance liquid chromatography (HPLC), and one or more of compounds of Formula (II), Formula (III), and Formula (IV), wherein each of the compound of Formula (II), Formula (III), or Formula (IV) is present, in an amount of 0.15% or less by weight, relative to the compound of Formula (I), when measured by HPLC,

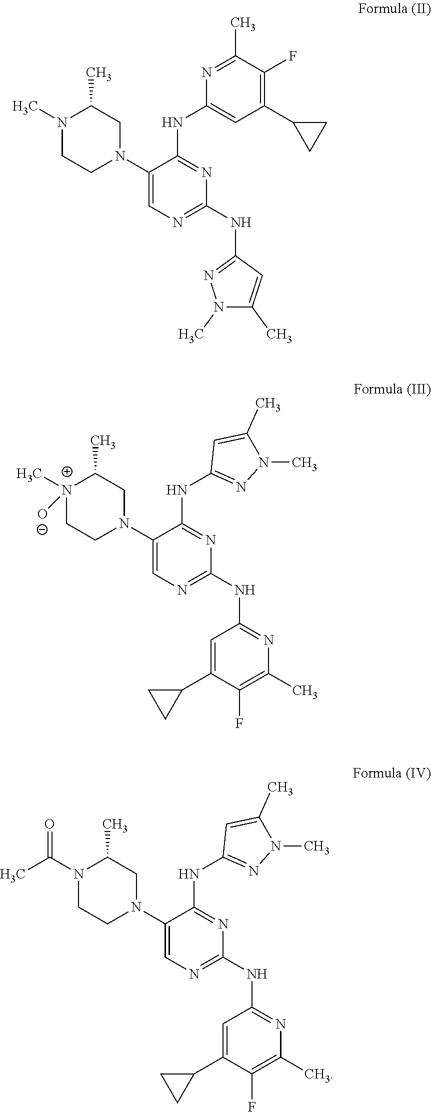

3. The composition according to claim 2, wherein each of the compounds of Formula (II), Formula (III), or Formula (IV), when present, is in a detectable amount of about 0.15% or less by weight, relative to the triaminopyrimidine compound of Formula (I), when measured by HPLC.

4. The composition according to claim 2, wherein each of the compounds of Formula (II), Formula (III), or Formula (IV) is present in the composition in a detectable amount by weight, when measured by HPLC.

5. The composition according to claim 2, wherein each of the compounds of Formula (II), Formula (III), or Formula (IV), is present, in an amount of about 0.11% or less by weight, relative to the compound of Formula (I), when measured by HPLC.

6. The composition according to claim 2, wherein the composition comprises triaminopyrimidine compound of Formula (I) having a purity of about 99.5% or more, when measured by area percentage of HPLC.

7. The composition according to claim 2, wherein each of the compounds of Formula (II), Formula (III), or Formula (IV), is present in an amount in the range of about 0.005% to about 0.15% by weight, relative to the compound of Formula (I), when measured by HPLC.

8. The composition according to claim 7, wherein each of the compounds of Formula (II), Formula (III), or Formula (IV), is present in an amount in the range of about 0.005% to about 0.11% by weight, relative to the compound of Formula (I), when measured by HPLC.

9. The composition according to claim 8, wherein each of the compounds of Formula (II), Formula (III), or Formula (IV), is present in an amount in the range of about 0.01% to about 0.11% by weight, relative to the compound of Formula (I), when measured by HPLC.

10. The composition according to claim 2, wherein the composition further comprises one or more compounds of Formula (V), or Formula (VI), each present, in an amount of about 500 ppm or less when measured by HPLC,

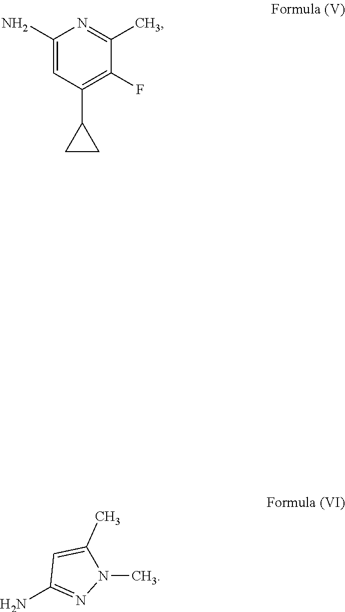

11. The composition according to claim 10, wherein each of the compounds of Formula (V), or Formula (VI) is present in an amount of about 134 ppm or less when measured by HPLC.

12. The composition according to claim 11, wherein each of the compounds of Formula (V) or Formula (VI) is present in an amount of about 2.0 ppm to about 134 ppm, when measured by HPLC.

13. The composition according to claim 12, wherein each of the compounds of Formula (V) or Formula (VI) is present in an amount of about 4 ppm to about 134 ppm, when measured by HPLC.

14. The composition according to claim 11, wherein each of the compounds of Formula (V), or Formula (VI), when present, is in a detectable amount of about 134 ppm or less, when measured by HPLC.

15. The composition according to claim 10, wherein each of the compounds of Formula (V) or Formula (VI) is present in a non-detectable amount, when measured by HPLC.

16. The composition according to claim 10, wherein the composition is stable for at least 6 months, and wherein each of the compounds of Formula (II), Formula (III), or Formula (IV) is present, in an amount of about 0.15%, or less, by weight, relative to the triaminopyrimidine compound of Formula (I), when measured by HPLC, and each of the compounds of Formula (V), or Formula (VI) is present in an amount of about 134 ppm or less, when measured by HPLC, when the composition is stored for at least 6 months at 40° C.±2° C. temperature and 75%±5% relative humidity, or at 25° C.±2° C. temperature and 60%±5% relative humidity.

17. The composition according to claim 16, wherein each of the compounds of Formula (II), Formula (III), or Formula (IV) is present, in an amount of about 0.11%, or less, by weight, relative to the triaminopyrimidine compound of Formula (I), when measured by HPLC.

18. A composition comprising a triaminopyrimidine compound of Formula (I), 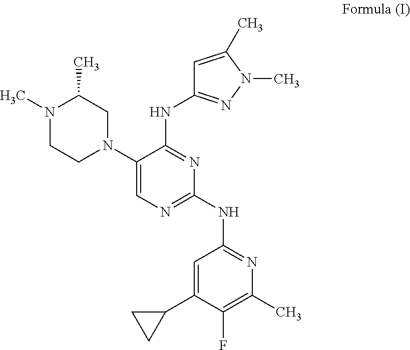
and one or more compounds of Formula (V), or Formula (VI), wherein each of the compounds of Formula (V), or Formula (VI) is present in an amount of about 500 ppm or less, when measured by HPLC,

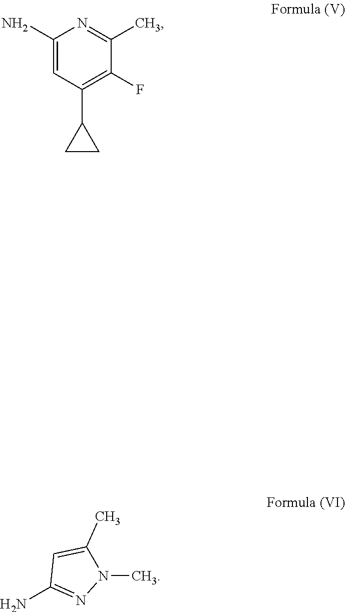

19. The composition according to claim 18, wherein each of the compounds of Formula (V) or Formula (VI) is present in an amount of about 134 ppm or less, when measured by HPLC.

20. A pharmaceutical composition comprising the composition according to claim 10, together with one or more pharmaceutically acceptable excipients.
